(19) United States
(12) Patent Application Publication
(10) Pub. No.: US 2016/0103096 A1
(43) Pub. Date: Apr. 14, 2016

(54) BIOCHEMICAL TEST CHIP AND METHOD FOR MANUFACTURING THE SAME

(71) Applicant: APEX BIOTECHNOLOGY CORP., Hsinchu (TW)

(72) Inventors: Mon-Wen Yang, Hsinchu (TW); Ying-Che Huang, Hsinchu (TW); Yun-Chung Shen, Hsinchu (TW)

(21) Appl. No.: 14/882,466

(22) Filed: Oct. 14, 2015

(30) Foreign Application Priority Data
Oct. 14, 2014 (TW) 103135515

(51) Int. Cl. G01N 27/416; H05K 3/10; G01N 33/487; G01N 27/327; G01N 27/30

(52) U.S. Cl. CPC G01N 27/4166; G01N 27/3275; G01N 27/301; G01N 33/48707; H05K 3/10

(57) ABSTRACT

Provided is a biochemical test chip including an insulating substrate, an electrode unit, a first insulating septum, a reactive layer and a second insulating septum. The insulating substrate has a first vent hole. The electrode unit is located on the insulating substrate. The first insulating septum is located on the electrode unit. The first insulating septum has an opening which exposes a part of the electrode unit. The reactive layer is located in the opening. The second insulating septum is located on the first insulating septum. The second insulating septum has a second vent hole. The first vent hole is at least partially overlapped with the second vent hole.

### CROSS-REFERENCE TO RELATED APPLICATION

This application claims the priority benefit of Taiwan application no. 103135515, filed on Oct. 14, 2014. The entirety of the above-mentioned patent application is hereby incorporated by reference herein and made a part of this specification.

### BACKGROUND OF THE DISCLOSURE

1. Field of the Disclosure

The disclosure relates to a biochemical test chip and a manufacturing method thereof, and particularly to a biochemical test chip capable of effectively preventing liquid sample overflow and a method for manufacturing the same.

2. Description of Related Art

Biochemical chips refer to the components that use molecular biology, analytical chemistry, biochemical reactions and other principles combined with microelectromechanical system (MEMS) technology and have advantages of being small and compact and capable of rapidly and parallelly processing a large number of biochemical sensing and reaction. With the increasing of advances in medicine and modern concept of health care, fast, inexpensive, small, and self-testing products that can be operated without professional operators (such as blood glucose meters, electronic ear thermometer and electronic sphygmomanometers, etc.) become more and more of concern. In this field, using biochemical test chips is already an extremely sophisticated technology, and analysis of blood glucose is the most widely used application.

As shown in FIG. 1, the U.S. Pat. No. 5,120,420 discloses a biochemical test chip. When the liquid sample is brought into contact with the sampling port 38, a tube-shaped space is formed among the upper cover 50, the opening 32 of the middle separating plate 30 and the insulating substrate 10. The combination of the tube-shaped space and the vent hole 55 of the upper cover 50 is then formed a sampling space 40 having a capillarity, such that the liquid sample L may flow to the end of the internal side of the opening 32 due to the difference between cohesive force and adhesive force. The air which had originally occupied the internal side of the opening 32 escapes from the vent hole 55 to the outside of the upper cover 50 due to the push of the liquid sample L, thereby generating inertia tension of air and liquid sample L, but also increasing the power of liquid sample L moving forward to the internal side of the opening 32. When the liquid sample L reaches the place where the vent hole 55 of the internal side of the opening 32 is located, at this time the liquid sample L fills to full around the vent hole 55, such that the liquid sample L is transformed from the original horizontal capillarity into vertical capillarity. In other words, through cohesive force and adhesive force generated around the vent hole 55 of the upper cover 50, the liquid sample L moves toward the vent hole 55 and further overflows to the outside of the upper cover 50. As such, the biochemical test chip is easy to induce measurement error and pollution problems.

Furthermore, the U.S. Pat. No. 5,997,817 discloses another type of biochemical test chip which includes an insulating substrate, an electrode system, a middle separating plate and an upper septum, and the difference between the biochemical test chip of the U.S. Pat. No. 5,997,817 and that of the U.S. Pat. No. 5,120,420 merely is that the vent hole is disposed on the insulating substrate in the U.S. Pat. No. 5,997,817. However, when the liquid sample fills around the vent hole, overflow of the liquid sample still occurs.

Moreover, the Taiwan Utility Model Patent (Patent No. M312667) discloses a biochemical test chip, wherein after the structures such as the insulating substrate, the middle separating plate, the upper septum, and the like, are assembled, by using one-time production method a vent hole may be formed on the insulating plate, the middle separating plate and the upper septum, thus it is no need to form the vent hole in advance before assembling, and thereby the assembling steps such as precisely aligning step can be omitted. The overflow of the liquid sample may be prevented in the Taiwan Utility Model Patent (Patent No. M312667), but dry enzyme is further disposed in the recess of the middle separating plate of the biochemical test chip, and the dry enzyme may be damaged during the vent hole forming process due to vibration, and measurement error may be induced.

### SUMMARY OF THE DISCLOSURE

The disclosure provides a biochemical test chip which is capable that liquid sample overflow and thereby inducing measurement error and pollution problems are effectively prevented.

The disclosure provides a biochemical test chip including an insulating substrate, an electrode unit, a first insulating septum, a reactive layer and a second insulating septum. The insulating substrate has a first vent hole. The electrode unit is located on the insulating substrate. The first insulating septum is located on the electrode unit. The first insulating septum has an opening which exposes a part of the electrode unit. The reactive layer is located in the opening. The second insulating septum is located on the first insulating septum. The second insulating septum has a second vent hole. The first vent hole is at least partially overlapped with the second vent hole.

According to an exemplary embodiment of the disclosure, the first vent hole is disposed in the insulating substrate at a first side of the opening, and the second vent hole is disposed in the second insulating septum at the first side of the opening.

According to an exemplary embodiment of the disclosure, the distance between the first vent hole and the first side of the opening is larger than the distance between the second vent hole and the first side of the opening.

According to an exemplary embodiment of the disclosure, the distance between the first vent hole and the first side of the opening is smaller than the distance between the second vent hole and the first side of the opening.

According to an exemplary embodiment of the disclosure, the second insulating septum further includes an inner claw structure disposed around the inner side of the second vent hole.

According to an exemplary embodiment of the disclosure, the shapes of the first vent hole and the second vent hole are polygonal.

According to an exemplary embodiment of the disclosure, the shapes of the first vent hole and the second vent hole are the same.

According to an exemplary embodiment of the disclosure, the shapes of the first vent hole and the second vent hole are different.

According to an exemplary embodiment of the disclosure, the inner side surface of the second insulating septum further includes a hydrophilic material.

A method for manufacturing a biochemical test chip is provided, and the steps are as follows. An insulation substrate is provided. The insulating substrate has a first vent hole. An electrode unit is formed on the insulating substrate. A first insulating septum covers the electrode unit. The first insulating septum has an opening. The opening exposes a part of the electrode unit. A reactive layer is formed in the opening. A second insulating septum covers the first insulating septum. The second insulating septum has a second vent hole. The first vent hole is at least partially overlapped with the second vent hole.

According to an exemplary embodiment of the disclosure, the first vent hole is disposed in the insulating substrate at the first side of the opening. The second vent hole is disposed in the second insulating septum at the first side of the opening.

According to an exemplary embodiment of the disclosure, the distance between the first vent hole and the first side of the opening is larger than the distance between the second vent hole and the first side of the opening.

According to an exemplary embodiment of the disclosure, the distance between the first vent hole and the first side of the opening is smaller than the distance between the second vent hole and the first side of the opening.

According to an exemplary embodiment of the disclosure, the method further includes forming an inner claw structure around the inner side of the second vent hole.

According to an exemplary embodiment of the invention, a method of forming the inner claw structure includes mechanical perforation.

According to an exemplary embodiment of the disclosure, the shapes of the first vent hole and the second vent hole are polygonal.

According to an exemplary embodiment of the disclosure, the shapes of the first vent hole and the second vent hole are the same.

According to an exemplary embodiment of the disclosure, the shapes of the first vent hole and the second vent hole are different.

According to an exemplary embodiment of the disclosure, coating a hydrophilic material on the inner side surface of the second insulating septum.

In light of the above, through the first vent hole in the insulating substrate being at least partially overlapped with the second vent hole in the second insulating septum, such that a side wall of the biochemical test chip of the disclosure, which is like one of the side walls of the sampling space 40 having capillarity of the conventional technique, has been damaged. As such, in the biochemical test chip of the disclosure, the vertical capillarity of the second vent hole can be avoided and the liquid sample overflows to the outside of the second insulating septum can further be prevented. Therefore, the disclosure provides a biochemical test chip which is capable to solve the induced measurement error and pollution problems that caused by liquid sample overflow.

To make the above features and advantages of the disclosure more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic cross-sectional view of a conventional biochemical test chip.

FIG. 2 is an exploded schematic view of a biochemical test chip according to one embodiment of the disclosure.

FIG. 3A is a schematic cross-sectional view taken along a line A-A′ in FIG. 2.

FIG. 3B through FIG. 3C are schematic cross-sectional views taken along a line A-A′ of a biochemical test chip according to another embodiment of the disclosure.

FIG. 4A through FIG. 4C are schematic top views of a biochemical test chip according to another embodiment of the disclosure.

FIG. 5 is a flowchart illustrating a manufacturing method of a biochemical test chip according to an embodiment of the disclosure.

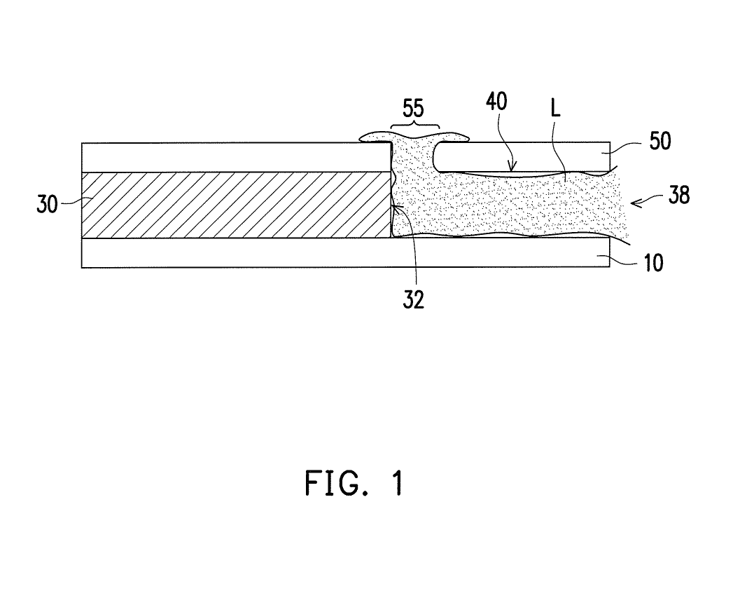

### DESCRIPTION OF THE EMBODIMENTS

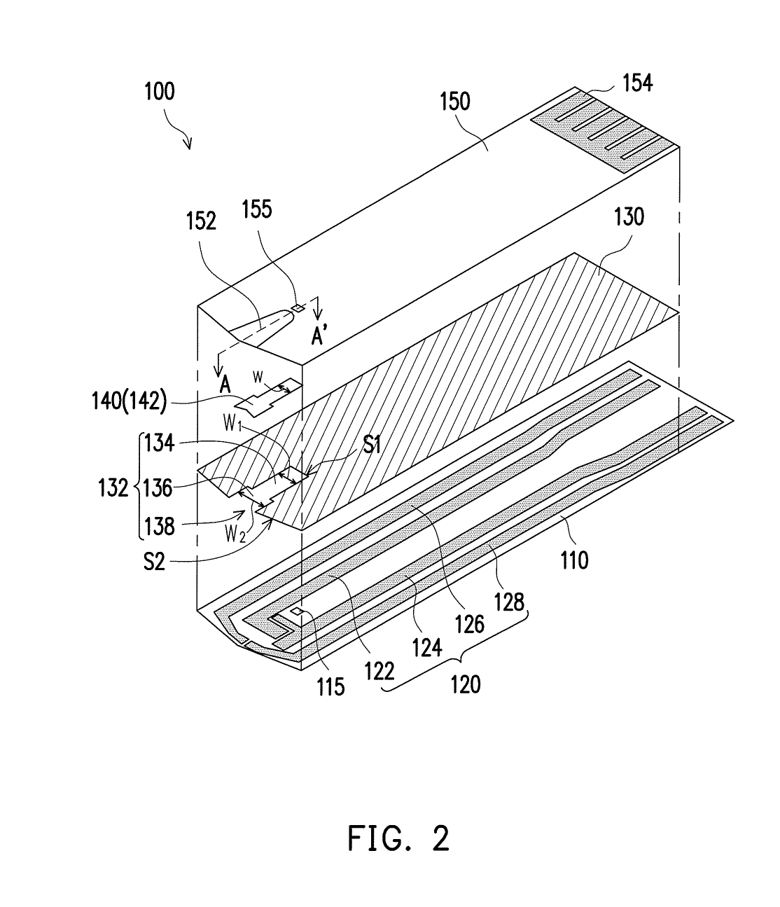

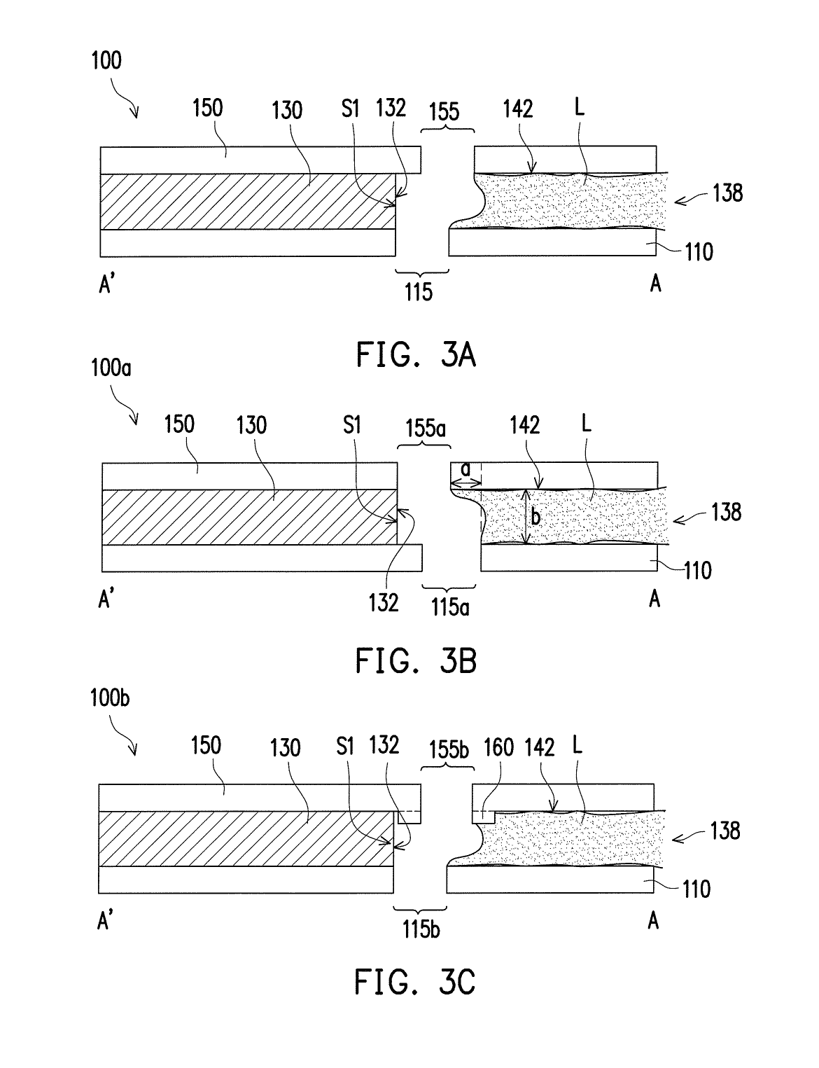

FIG. 2 is an exploded schematic view of a biochemical test chip according to one embodiment of the disclosure. FIG. 3A is a schematic cross-sectional view taken along a line A-A′ in FIG. 2. FIG. 3B through FIG. 3C are schematic cross-sectional views taken along a line A-A′ of a biochemical test chip according to another embodiment of the disclosure. For the sake of the drawings being brief and clear, the line A-A′ is only shown on the second insulating septum 150 in FIG. 2, but the cross-sectional views taken along the line A-A′ shown in FIG. 3A through FIG. 3C are cross-sectional views illustrating from the second insulating septum 150 to the insulating substrate 110.

Referring to FIG. 2, FIG. 3A and FIG. 3B, the disclosure provides a biochemical test chip 100 including an insulating substrate 110, an electrode unit 120, a first insulating septum 130, a reactive layer 140 and a second insulating septum 150. In the present embodiment, the biochemical test chip 100 is an electrochemical test chip for receiving a user's blood sample, and used for measuring the value of blood glucose, cholesterol, uric acid, lactic acid, hemoglobin, etc. in the blood. However, the disclosure is not limited thereto. In other embodiments, the biochemical test chip 100 may also be used in any kind of liquid sample, as long as capable of producing electrochemical reaction with the reactive layer 140 or having ability of specifically identifying biological material or signal.

The insulating substrate 110 is a substrate which has an even surface and electrically insulation, and is endurable to a temperature between 40° C. and 120° C. In one embodiment, the material of the insulating substrate 110 includes polyvinyl chloride (PVC), glass fiber (FR-4), polyester suphone, bakelite, polyethylene terephthalate (PET), polycarbonate (PC), polypropylene (PP), polyethylene (PE), polystyrene (PS), glass plate, ceramic, or any combination of these materials. Certainly, the material of the insulating substrate 110 is not limited thereto.

As shown in FIG. 2, the electrode unit 120 is located on the insulating substrate 110. The electrode unit 120 includes a work electrode 122, a reference electrode 124 and identification electrodes 126, 128, which are insulated from one another. In the present embodiment, the identification electrodes 126, 128 are disposed at the outer sides of the work electrode 122 and the reference electrode 124. However, the disposing of the electrode unit 120 may be altered according to various requirements, the arrangement of the electrode unit is not limited, the number of electrodes is not limited, the designer may alter the number of electrodes according to actual requirements, and the disclosure is not limited thereto.

In the present embodiment, the identification electrodes 126, 128 may be conducted through the liquid sample L which enters from the sampling port 138 in the subsequent manufacturing process, and thereby actuating the measuring steps. The work electrode 122 and the reference electrode 124 are used for determining whether the liquid sample L which enters during the subsequent manufacturing process produces electrochemical reaction with the reactive layer 140 or not, or whether produces a specific identification biological signal or not. However, the disclosure is not limited thereto, in another embodiment, the electrodes 126, 128 may also be used for measuring the disruptors. For instance, when the electrodes 122, 124 measure the blood glucose, the value of blood glucose can be calibrated by using the measurement value of the disruptors. On the other hand, in other embodiments, it is also possible that the electrodes 126, 128 are used for detecting a first sample concentration, and the electrodes 122, 124 are used for detecting a second sample concentration. The material of the electrode unit 120 may be any conductive material, such as palladium gum, gum platinum, gold plastic, titanium plastic, carbon plastic, silver plastic, copper plastic, mixture of gold and silver plastic, mixture of carbon and silver plastic, or any combination of these conductive materials. In one embodiment, the electrode unit 120 is composed of a conductive carbon powder layer. In another embodiment, the electrode unit 120 is composed of a metal layer. And in another embodiment, the electrode unit 120 is composed of a conductive silver plastic layer and a conductive carbon powder layer located thereon, wherein generally the impedance of the conductive carbon powder layer is much larger than that of the conductive silver plastic layer or other metal plastic layer.

The first insulating septum 130 is located on the electrode unit 120. The first insulating septum 130 has an opening 132, and the opening 132 exposes at least a part of the work electrode 122 and the reference electrode 124. Specifically, the opening 132 includes a first region 134, a second region 136, and a sampling port 138. The first region 134 is located at the first side S1 of the opening 132; the sampling port 138 is located at the second side S2 of the opening 132; and the second region 136 is located between the first region 134 and the sampling port 138. In the present embodiment, the disclosure does not limit area and shape of the opening 132, as long as the opening 132 exposes a part of the work electrode 122 and a part of the reference electrode 124 which are required for measurement. In one embodiment, the material of the first insulating septum 130 may include, but not limited to, PVC insulating tape, ethylene terephthalate insulating tape, thermal drying insulating paint or UV-curable insulating paint.

The reactive layer 140 is located in the opening 132. The reactive layer 140 covers at least the work electrode 122 and the reference electrode 124 of the opening 132, so as to perform an electrochemical reaction or to produce a specific identification biological signal. The reactive layer 140 at least includes an active material and a conductive medium, for the liquid sample L (may be, for example, blood) to produce a chemical reaction. In general, the area of the reactive layer 140 is smaller than or equal to the area of the opening 132, and the shape thereof is not limited as long as the reactive layer 140 can produce a chemical reaction with the liquid sample L. In one embodiment, the active material includes immobilized enzyme or enzyme which is not immobilized. For example, the active material includes glucose oxidase, antigens, antibodies, microbial cells, plant and animal cells, plant and animal tissue, which have biological identification ability. The conductive medium is used for receiving the electrons generated after the reaction between the active material and the blood sample, and conducting the electrons to the biometry through the electrode unit. The composition thereof may be, but not limited to, enzyme (e.g., glucose glucoamylase), conductive medium (e.g., ferricyanide salt), phosphate buffer, protecting agent (such as: protein, dextrin, dextran, amino acids, etc.).

The second insulating septum 150 is located on the first insulating septum 130 and the reactive layer 140. Since the second insulating septum 150 entirely covers on the reactive layer 140, the above, below and three sidewalls of the reactive layer 140 (except the sampling port 138) are surrounded by the second insulating septum 150, the insulating substrate 110 and the first insulating septum 130 and form a tube-shaped space. When the liquid sample L enters the tube-shaped space, the adhesive force of the liquid sample L in the tube-shaped space is larger than the cohesive force of the liquid sample L, such that the liquid sample L may proceed forward. At this time, the liquid sample L may be brought into contact with the reactive layer 140 which is in the tube-shaped space, such that the liquid sample L is mixed with the active material and the conductive medium in the reactive layer 140, so as to form a reactive region 142 in the tube-shaped space (as shown in FIG. 3A). In the present embodiment, the width W₁ of the first region 134 may be smaller than the width W₂ of the second region 136, so that the liquid sample L may be rapidly filled in the first region 134 and the second region 136, in order to facilitate subsequent electrochemical reaction. However, the disclosure is not limited thereto, in other embodiments, the width W₁ of the first region 134 may be equal to the width W₂ of the second region 136.

In addition, in order the user may see the status of the liquid sample L injecting into the reactive region 142, in the present embodiment, the second insulating septum 150 has a transparent observing region 152. The transparent observing region 152 exposes at least a part of the reactive region 142, in order to facilitate to observe the status of liquid sample L injecting into the reactive region 142. For instance, if the user observes from the transparent observing region 152 that the liquid sample L has fully filled, it represents that the volume of the liquid sample L is enough and no need to inject liquid sample L. On the contrary, if the user observes from the transparent observing region 152 that the liquid sample L does not fully fill and a blank yet exists, then the user may continue to provide liquid sample L. Certainly, the shape of the transparent observing region 152 of the second insulating septum 150 is not limited to the abovementioned design, it can be designed according to actual requirement.

In the present embodiment, the second insulating septum 150 further includes an identification unit 154, and the identification unit 154 is located at an end, which is away from the transparent observing region 146, of the second insulating septum 150. The identification unit 154 includes a plurality of electrical components, wherein the disposing locations, numbers and shapes of the electrical components may be used for identifying the type of the biochemical test chip 100, and corresponding relative calibration parameters or models are employed to perform measurement. In other words, the numbers and locations of the electrical components are used for determining an identification code of the biochemical test chip 100, so that based on this, for example, the biometry may identify the type of the biochemical test chip 100.

The electrical components may be any kind of electrical components having conductivity, for example, electrical components having electrical characteristic of passive elements. In one embodiment, the electrical components may be a resister, the material thereof is the same as the electrode unit, and the forming method may be the techniques such as screen printing, imprinting, thermal transfer printing, spin coating, inkjet printing, laser ablation, deposition, electroplating, and the like. In another embodiment, the electrical components included in the identification unit 154 may also be resistors, capacitors, inductors, and/or combinations thereof.

It should be noted that, as shown in FIG. 2, the insulating substrate 110 has a first vent hole 115. The first vent hole 115 is disposed in the insulating substrate 110 at the first side S1 of the opening 132, namely, located at an end of the reactive region 142 in the first insulating septum 130 and overlapped with the opening 132 (as shown in FIG. 3A). The second insulating septum 150 has a second vent hole 155, wherein the second vent hole 155 is disposed in the second insulating septum 150 at the first side S1 of the opening 132, namely, located at an end of the reactive region 142 in the first insulating septum 130 and overlapped with the opening 132. The first vent hole 115 and the second vent hole 155 are used for discharge the air within the reactive region 142, so as to prevent the liquid sample L from being blocked by the bubbles and unable to successfully move forward smoothly in the reaction region 142.

In the following embodiment and drawings, the same or like numbers stand for the same or like elements for simple illustration. For instance, the first vent hole 115 and the first vent hole 215a, 215b, 215c are the same or similar elements, and it is not repeated herein.

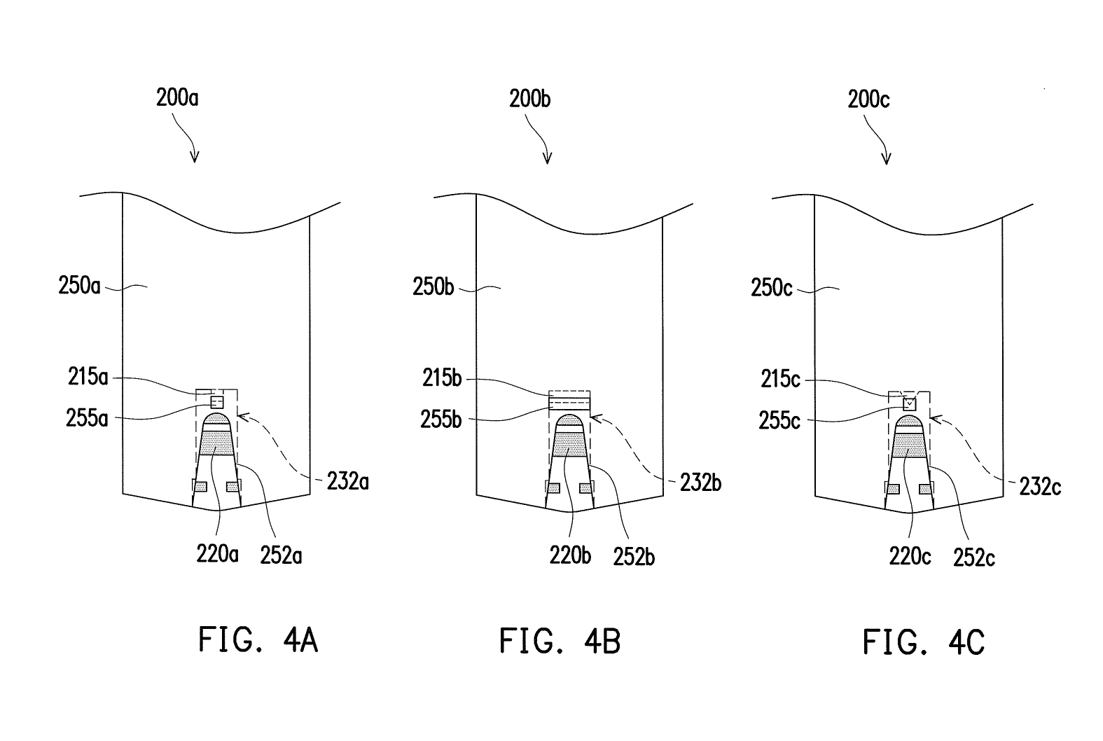

The shapes of the first vent hole 115 and the second vent hole 155 are not limited in the disclosure, in the present embodiment, the shapes of the first vent hole 115 and the second vent hole 155 are polygonal, and may be square, rectangular, circular, elliptical, or triangular, etc. The following takes one of them as an example for reference. FIG. 4A through FIG. 4C are schematic top views of a biochemical test chip according to another embodiment of the disclosure. As shown in FIG. 4A through FIG. 4C, both of the shapes of the first vent hole 215a and the second vent hole 225a are square, and both of them are located at the end of the opening 232a and at least partially overlapped. As shown in FIG. 4B, both of the shapes of the first vent hole 215b and the second vent hole 225b are rectangular, and each has a width which is equal to the width of the opening 232b. The first vent hole 215b and the second vent hole 255b are located at the end of the opening 232b and at least partially overlapped. As shown in FIG. 4C, the shape of the first vent hole 215c is triangular, the shape of the second vent hole 255c is square, and an angle of the triangular first vent hole 215c and a side of the square second vent hole 255c are at least partially overlapped.

In addition, a hydrophilic material (not shown in the drawings) may be coated on the lower surface, which is located in the reactive region 142, of the second insulating septum 150, so as to strengthen the capillary action of the inner sidewalls of the reactive region 142, such that the liquid sample L may be guided into the reactive region 142 rapidly and effectively.

Referring to FIG. 3A, in the present embodiment, the first vent hole 115 and the second vent hole 155 are at least partially overlapped so as to form a cliff, and the distance between the first vent hole 115 and the first side S1 of the opening 132 is smaller than the distance between the second vent hole 155 and the first side S1 of the opening 132. In other words, the second vent hole 155 is nearer to the sampling port 138 than the first vent hole 115, therefore after the liquid sample L enters the reactive region 142 through the sampling port 138, the liquid sample L may first reach the edge side of the second vent hole 155. Since the cliff has damaged one sidewall in the reactive region 142, the liquid sample L has no other tube wall to adhere, resulting that the cohesive force of the liquid sample L is larger than the adhesive force between the insulating substrate 110 and the second insulating septum 150. Additionally, since the liquid sample L does not have a third tube wall to adhere, generating a vertical capillarity can be avoided and the liquid sample L overflow to the first vent hole 115 and the second vent hole 155 may be prevented. As such, the liquid sample L of the biochemical test chip 100 of the present embodiment may stop flowing at the edge side of the second vent hole 155.

In another embodiment, as shown in FIG. 3B, the first vent hole 115a and the second vent hole 155a are at least partially overlapped so as to form a cliff, and the distance between the first vent hole 115a and the first side S1 of the opening 132 is larger than the distance between the second vent hole 155a and the first side S1 of the opening 132. In other words, the first vent hole 115a is nearer to the sampling port 138 than the second vent hole 155a, therefore after the liquid sample L enters the reactive region 142 through the sampling port 138, the liquid sample L may first reach the edge side of the first vent hole 115a. At this time, besides the cohesive force and the adhesive force, the force of gravity may also affect the liquid sample L. Since the directions of the cohesive force and the force of gravity are opposite, if the cohesive force of the liquid sample L is larger than the force of gravity, then the liquid sample L may not overflow. The calculation of reaction force F of the force of gravity exerting to the liquid sample L is as follows:

F=1/2abwρg

a=extending length

b=height of the reactive region

w=width of the reactive region

ρ=density of the liquid sample

g=force of gravity (9.8 m/s²)

In general, the strength of the reactive force of the force of gravity exerting to the liquid sample L may be controlled by the extending length a. The smaller the extending length a is, the less the overflow of the liquid sample L would be. In one embodiment, the extending length a is smaller than 3 mm. In another embodiment, the extending length a is 1 mm. In another embodiment, the extending length a is 0.5 mm.

In another embodiment, as shown in FIG. 3C, the first vent hole 115b and the second vent hole 155b are at least partially overlapped so as to form a cliff, and the second vent hole 155b is nearer to the sampling port 138 than the first vent hole 115b, substantially, similar to the structure of the biochemical test chip 100 shown in FIG. 3A. The difference is that the second insulating septum 150 of the biochemical test chip 100b in FIG. 3C has an inner claw structure 160. The inner claw structure 160 is disposed around the inner side of the second vent hole 155b. When the liquid sample L enters the reactive region 142 through the sampling port 138, not only capillarity may stop to generate due to the cliff, but also the inner claw structure 160 may exert a gripping force to the liquid sample L to increase the cohesive force, such that the liquid sample L may remain in the reaction region 142. As such, as illustrated in another embodiment of the disclosure, the liquid sample L may be effectively locked at the end of the reactive region 142, and the effect of preventing the liquid sample L from overflowing to the outside of the second insulating septum 150 may be achieved.

It should to be mentioned that the second vent hole 155b of the second insulating septum 150 may be formed by mechanical perforation method. This method not only forms the second vent hole 155b, but also simultaneously may form the inner claw structure 160 around the inner side of the second vent hole 155b. Thus, besides preventing the liquid sample L overflow, the effect of simplifying the manufacturing process and reducing the manufacturing cost is achieved.

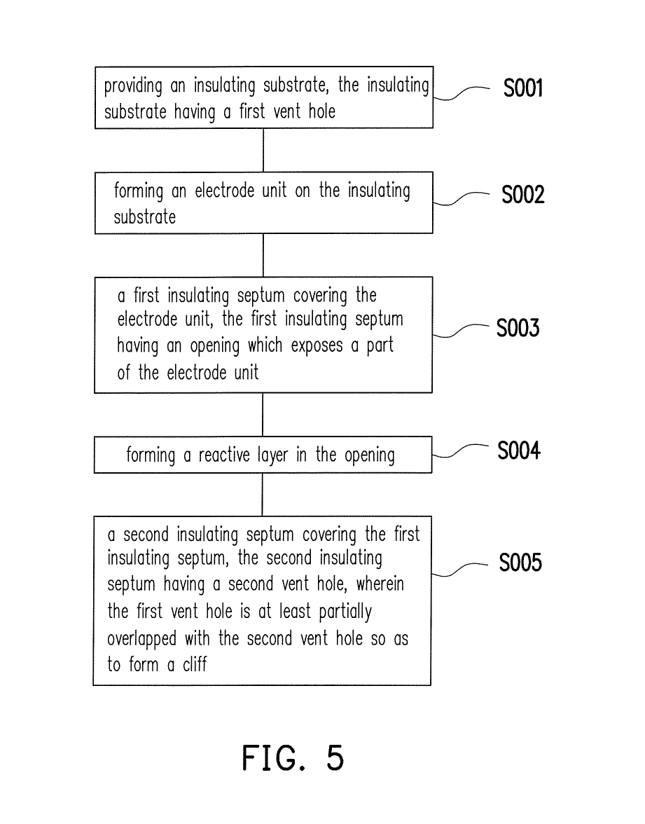

FIG. 5 is a flowchart illustrating a manufacturing method of a biochemical test chip according to an embodiment of the disclosure.

A method for manufacturing a biochemical test chip is provided, and the steps are as follows. First, in the step S001, an insulating substrate is provided, wherein the insulating substrate has a first vent hole. Next, in the step S002, an electrode unit is formed on the insulating substrate, wherein the electrode unit includes a work electrode, a reference electrode and identification electrodes, which are insulated from one another. The identification electrodes may be disposed at the outer sides of the work electrode and the reference electrode. Then, a first insulating septum covers the electrode unit (as illustrated in the step S003). The first insulating septum has an opening. The opening exposes a part of the electrode unit, namely, at least exposes the work electrode and the reference electrode. Referring to the step S004, a reactive layer is formed in the opening. Next, a second insulating septum covers the first insulating septum (as illustrated in the step S005). The second insulating septum has a second vent hole, wherein the first vent hole and the second vent hole are located at an end of the opening, i.e., the end of the reactive layer. The first vent hole is at least partially overlapped with the second vent hole, in order to form a cliff. The cliff has an effect of preventing the liquid sample injected in the subsequent process from overflow. Moreover, besides the second insulating septum has the second vent hole, it also has an inner claw structure disposed around the inner side of the second vent hole. The inner claw structure may further lock the liquid sample effectively to remain at the end of the reactive layer, and further can prevent the liquid sample injected in the subsequent process from overflow.

In light of the foregoing, in the disclosure, the first vent hole is at least partially overlapped with the second vent hole, in order to form a cliff. Through this configuration, the cliff may damage a side wall in the reactive region, such that the liquid sample does not have other tube wall to adhere, thus the liquid sample may stop flowing at the edge sides of the first vent hole and the second vent hole. As such, in the biochemical test chip of the disclosure, the vertical capillarity of the second vent hole can be avoided and the liquid sample overflows to the outside of the second insulating septum can further be prevented. Furthermore, precisely alignment is unnecessary as long as the first vent hole is at least partially overlapped with the second vent hole, thus the disclosure achieves an effect of simplifying the manufacturing method of the biochemical test chip. Additionally, the second insulating septum of another embodiment further includes an inner claw structure disposed around the inner side of the second vent hole. Therefore, in the disclosure, not only the cliff stops the generating of capillarity, but also the inner claw structure may apply a gripping force to the liquid sample in order to increase the cohesive force of the liquid sample, and may effectively facilitate the liquid sample to remain in the reactive region, thereby the measurement error and pollution problems of the biochemical test chip are solved.

Although the disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and not by the above detailed descriptions.

What is claimed is:

1. A biochemical test chip, comprising:
an insulating substrate, having a first vent hole;
an electrode unit, located on the insulating substrate;
a first insulating septum, located on the electrode unit and having an opening, wherein the opening exposes a part of the electrode unit;
a reactive layer, located in the opening; and
a second insulating septum, located on the first insulating septum and having a second vent hole, wherein the first vent hole is at least partially overlapped with the second vent hole.

2. The biochemical test chip as claimed in claim 1, wherein the first vent hole is disposed in the insulating substrate at a first side of the opening, and the second vent hole is disposed in the second insulating septum at the first side of the opening.

3. The biochemical test chip as claimed in claim 2, wherein a distance between the first vent hole and the first side of the opening is larger than a distance between the second vent hole and the first side of the opening.

4. The biochemical test chip as claimed in claim 2, wherein a distance between the first vent hole and the first side of the opening is smaller than a distance between the second vent hole and the first side of the opening.

5. The biochemical test chip as claimed in claim 1, wherein the second insulating septum further comprises an inner claw structure disposed around an inner side of the second vent hole.

6. The biochemical test chip as claimed in claim 1, wherein shapes of the first vent hole and the second vent hole are polygonal.

7. The biochemical test chip as claimed in claim 1, wherein shapes of the first vent hole and the second vent hole are the same.

8. The biochemical test chip as claimed in claim 1, wherein shapes of the first vent hole and the second vent hole are different.

9. The biochemical test chip as claimed in claim 1, wherein an inner side surface of the second insulating septum further comprises a hydrophilic material.

10. A manufacturing method of a biochemical test chip, the method comprising:
providing an insulating substrate, the insulating substrate having a first vent hole;
forming an electrode unit on the insulating substrate;
a first insulating septum covering the electrode unit, wherein the first insulating septum has an opening, and the opening exposes a part of the electrode unit;
forming a reactive layer in the opening; and
a second insulating septum covering the first insulating septum, the second insulating septum having a second vent hole, wherein the first vent hole is at least partially overlapped with the second vent hole.

11. The manufacturing method of the biochemical test chip as claimed in claim 10, wherein the first vent hole is disposed in the insulating substrate at a first side of the opening, and the second vent hole is disposed in the second insulating septum at the first side of the opening.

12. The manufacturing method of the biochemical test chip as claimed in claim 11, wherein a distance between the first vent hole and the first side of the opening is larger than a distance between the second vent hole and the first side of the opening.

13. The manufacturing method of the biochemical test chip as claimed in claim 11, wherein a distance between the first vent hole and the first side of the opening is smaller than a distance between the second vent hole and the first side of the opening.

14. The manufacturing method of the biochemical test chip as claimed in claim 10, further comprising forming an inner claw structure around an inner side of the second vent hole.

15. The manufacturing method of the biochemical test chip as claimed in claim 14, wherein a method for foaming the inner claw structure comprises a mechanical perforation.

16. The manufacturing method of the biochemical test chip as claimed in claim 10, wherein shapes of the first vent hole and the second vent hole are polygonal.

17. The manufacturing method of the biochemical test chip as claimed in claim 10, wherein shapes of the first vent hole and the second vent hole are the same.

18. The manufacturing method of the biochemical test chip as claimed in claim 10, wherein shapes of the first vent hole and the second vent hole are different.

19. The manufacturing method of the biochemical test chip as claimed in claim 10, further comprising coating a hydrophilic material on an inner side surface of the second insulating septum.
